(19) United States
(12) Patent Application Publication
(10) Pub. No.: US 2017/0105999 A1
(43) Pub. Date: Apr. 20, 2017

(54) METHOD FOR TREATING DRUG RESISTANT CANCER

(71) Applicant: NATIONAL YANG MING UNIVERSITY, Taipel (TW)

(72) Inventors: CHI-YING HUANG, Taipei (TW); PETER MU-HSIN CHANG, Taipei (TW); KUAN-YU CHEN, Taipei (TW); CHUN-HUNG WU, Taipei (TW); TAI-SHAN CHENG, Taipei (TW); CHENG-HAO YU, Taipei (TW)

(21) Appl. No.: 15/316,079

(22) PCT Filed: Jun. 2, 2015
(86) PCT No.: PCT/CN2015/000380

§ 371 Date: Dec. 2, 2016

Related U.S. Application Data
(60) Provisional application No. 62/006,630, filed on Jun. 2, 2014.

(51) Int. Cl. A61K 31/5415; A61K 33/24; A61K 31/7068; A61K 31/517; A61K 31/519; A61K 45/06; A61K 31/5377

(52) U.S. Cl. CPC A61K 31/5415; A61K 45/06; A61K 33/24; A61K 31/5377; A61K 31/517; A61K 31/519; A61K 31/7068

(57) ABSTRACT

The present invention relates to a method for treating a subject with a cancer resistant to a chemotherapeutic drug comprising administering to said subject a therapeutically effective amount of prochlorperazine or its analog or metabolite, or a pharmaceutically acceptable salt thereof, in combination of the chemotherapeutic drug. The present invention also relates to a method for preventing cancer metastasis with the combination of prochlorperazine in combination of a chemotherapeutic drug.

### CROSS REFERENCE

This application claims priority to U.S. Provisional Patent Appl. No. 62/006,630, filed on Jun. 2, 2014, the contents of which is incorporated by reference in its entirety.

### FIELD OF THE INVENTION

The present invention relates to a method for treating a drug resistant cancer with an anti-emetic drug in combination of a chemotherapeutic drug.

### BACKGROUND OF THE INVENTION

Chemotherapy, particularly with a combination of anti-cancer agents, is the treatment of choice for delocalized tumors that are untreatable by surgery or radiation. However, some patients relapse after even a short period of time, and do not respond to a second course of chemotherapy.

Most malignant tumors show some sensitivity to cytotoxic drugs. Accordingly, treatment with these drugs generally provides remission and shrinkage of the tumor that may last for weeks to months. Nonetheless, in many cases the tumors regrow, and this regrowth is resistant to further cytotoxic treatments.

Theoretically it should be possible to solve this problem by administering a combination of drugs that act differently. This method of treatment is based upon the extremely small probability that two or more different drug resistances would arise spontaneously in the same cell. Combination chemotherapy appeared to obviate the problem of drug-resistant tumor cells.

Research was then directed to protocols for administering anticancer drugs in combinations. Newly developed drugs and combination chemotherapy several decades ago produced high cure rates for some childhood leukemia and for Hodgkin's disease. However, the major killers, such as lung cancer, breast cancer, and cancers of the gastrointestinal tract, remained resistant to chemotherapy. The failures of combination chemotherapy were not understandable. Many theories were proposed to explain the observations, but few of these theories could be adequately tested.

Although significant strides have been made in the development of pharmaceutical compositions for treating cancer, new methods of treating drug resistant cancer are required.

### SUMMARY OF THE INVENTION

According to the invention, it was unexpectedly found that prochlorperazine in combination of a chemotherapeutic drug exhibits a synergistic effect in reducing the size and number of the cancer cells, and inhibiting the growth of cancer cells with drug resistance properties.

In one aspect, the invention provides a method for treating a subject with a cancer resistant to a chemotherapeutic drug. The method comprises administering to said subject a therapeutically effective amount of prochlorperazine or its analog or metabolite, or a pharmaceutically acceptable salt thereof, in combination of the chemotherapeutic drug.

In one further aspect, the invention provides a method for preventing cancer metastasis. The method comprises administering to a subject in need thereof a therapeutically effective amount of prochlorperazine or its analog or metabolite, or a pharmaceutically acceptable salt thereof, in combination of a chemotherapeutic drug

In one more aspect, the invention provides a pharmaceutical composition or combination for treating a subject with a cancer resistant to a chemotherapeutic drug or for preventing cancer metastasisc, comprising a therapeutically effective amount of prochlorperazine or its analog or metabolite, or a pharmaceutically acceptable salt thereof, in combination of a chemotherapeutic drug, in combination of a chemotherapeutic drug.

In one yet aspect, the invention provides a use of prochlorperazine or its analog or metabolite, or a pharmaceutically acceptable salt thereof, for manufacturing a medicament for treating a subject with a cancer resistant to a chemotherapeutic drug in combination of a chemotherapeutic drug.

In one further yet aspect, the invention provides a use of prochlorperazine or its analog or metabolite, or a pharmaceutically acceptable salt thereof, for manufacturing a medicament for preventing cancer metastasis in combination of a chemotherapeutic drug.

In one embodiment of the invention, the chemotherapeutic drug is selected from the group consisting of gefitinib, erlotinib, afatinib, pemetrexed, cisplatin, paclitaxel, docetaxel, gemcitabine, navelbine, irinotecan, avastin, 5-fluorouracil, methotrexate, oxaliplatin, tegafur-gimeracil-oteracil potassium (TS-1), and epidermal growth factor receptor (EGFR)-tyrosine kinase inhibitors and combination thereof.

### BRIEF DESCRIPTION OF THE DRAWINGS

The foregoing summary, as well as the following detailed description of the invention, will be better understood when read in conjunction with the appended drawing. In the drawings:

FIG. 1 showed that prochlorperazine induced apoptosis and synergistically enhanced cytotoxicity in combination of gemcitabine in CL152 spheres; wherein

FIG. 1(A) showed that CL152 spheres were collected and analyzed by flow cytometry after treatment with prochlorperazine for 48 hours. Prochlorperazine led to a dose-dependent increase in apoptosis.

FIG. 1(B) showed that prochlorperazine had synergistic effects with gemcitabine in CL152 spheres.

FIG. 1(C) showed that prochlorperazine inhibited cell migration after prochlorperazine treatment.

FIG. 1(D) showed that prochlorperazine increased the number of β-galactosidase positive cells and induced senescence at A549 cells treated with prochlorperazine for 24 hours. A549 cells were treated with 50 μM resveratrol as a senescent positive control.

FIG. 2 showed that prochlorperazine reduced the percentage of cancer stern-like cells and enhanced chemotherapeutic agents and gefitinib induced cytotoxicity; wherein

FIG. 2(A) showed that prochlorperazine decreased cell survival of CL141 cancer stem-like sphere cells and enhanced anti-cancer activity of pemetrexed;

FIG. 2(B) showed that prochlorperazine decreased cell survival of CL97 cancer stem-like sphere cells and enhanced anti-cancer activity of gefitinib;

FIG. 2(C) showed that prochlorperazine inhibited sphere formation activity of HCC827 cells and enhanced anti-cancer activity of cisplatin;

FIG. 2(D) showed that prochlorperazine inhibited sphere formation activity of H1299 cells and enhanced anti-cancer activity of cisplatin;

FIG. 3 provides that the in vivo monitoring of prochlorperazine-mediated antitumor effects either alone and in combination of a standard chemotherapeutic agent; wherein:

FIG. 3(A) showed that 5×10⁵ H441 bulk tumor cells were subcutaneously injected into the right flank of NOD/SCID mice which were subsequently divided into vehicle (control) and prochlorperazine (5 mg/kg/day, 5 times a week). Tumor burden was measured using a caliper and fold change in tumor size was plotted over time; our initial results demonstrated that at this concentration, prochlorperazine alone suppressed (or delayed) tumorigenesis in vivo. In subsequent experiments, we used prochlorperazine in combination of a standard chemotherapeutic agent for tumor suppressive effects.

FIG. 3(B) indicated that the combination of pemetrexed (1 mg/kg, 5 times a week) and prochlorperazine (1 mg/kg, 5 times a week) provided the most significant tumor suppressive effect as compared to control, pemetrexed alone (1 mg/kg, 5 times a week) and the combination of pemetrexed (1 mg/kg, 5 times a week) and cisplatin (1 mg/kg, twice a week).

FIG. 3(C) shows the results of the experiments on adenocarcinoma tumor model , wherein the standard chemotherapeutic regimen is the combination of pemetrexed (50 mg/kg) and cisplatin (3 mg/kg); and the results show that the addition of prochlorperazine (5 mg/kg) with the standard regimen yielded the least tumor burden followed by prochlorperazine alone (5 mg/kg), the combination of pemetrexed (50 mg/kg) and cisplatin (3 mg/kg) and the vehicle control.

FIG. 3(D) shows the results of the experiments on squamous model with a standard treatment with the combination of gemcitabine and cisplatin; indicating that the addition of prochlorperazine (5 mg/kg) to the standard treatment (the combination of 60 mg/kg gemcitabine and 3 mg/kg cisplatin) provided the most tumor suppressive effect followed by prochlorperazine alone (5 mg/kg), the combination of gemcitabine and cisplatin, and the vehicle control.

FIG. 3 (E) demonstrates the results of the treatment with gefitinib in addition of prochlorperazine on gefitinib-resistant NSCLC model; wherein the combination of gefitinib (100 mg/kg) and prochlorperazine (5 mg/kg) showed the highest degree of tumor inhibition followed by prochlorperazine alone (5 mg/kg); gefitinib alone (100 mg/kg) and the vehicle control groups showed similar tumor burden.

FIG. 4 provides some images showing the results of a case study using prochlorperazine for lung squamous cell carcinoma patient wherein the patient is a 81 y/o male with lung squamous cell carcinoma, right lower lung with right upper lung metastasis. The patient accepted radiation over right lower lung primary lesion due to hemoptysis; and then received Tarceva™ (containing erlotinib as active ingredient) since May 19, 2010. The condition kept stable and first CT showed a 0.5 cm right upper lung metastasis. This lesion progressed to 2 cm on May 20, 2013 with worsening cough and dyspnea. Due to old age and poor general condition, he decided to continue Tarceva™ with only adding prochlorperazine. After taking prochlorperazine, the cough and dyspnea improved and followed CT after 3 months showed stable disease. The regimen was continued until last followed-up CT on 2014/06/10 showed the tumor progression. The patient died at the end of 2014.

FIG. 5 provides some images showing the results of a case study using prochlorperazine for lung adenocarcinoma patient. Lung adenocarcinoma patient (50 y/o female, LUL) carried with EGFR-L858R mutation was first treated with gefitinib since 2012 (with malignant pleural effusion) and pemetrexed since Aug. 24, 2013. The patient was then took prochlorperazine along with pemetrexed on Nov. 11, 2013. The regimen was continued and last followed-up CT on Jul. 19, 2014, showing that the tumor and pleural effusion were reduced significantly. The patient still survives now.

FIG. 6 provides some images showing the results of a case study using prochlorperazine for signet ring cell carcinoma patient. Signet ring cell carcinoma patient (58 y/o female), with multiple intra-abdominal metastasis, which has average median survival 7-8 months, were treated with several kinds of chemotherapy (cisplatin, 5-fluorouracil, irinotecan, paclitaxel, gemcitabine, avastin, TS-1, oxaliplatin, paclitaxeli5-fluorouracil and avastin/gemcitabine/TS-1) since Jul. 16, 2012. Patient took prochlorperazine on Feb. 25, 2013. The regimen was continued and last followed-up CT on Jun. 18, 2014, showing that the tumor was stable. The patient died at the end of 2014.

FIG. 7A and 7B showed the clinical course and the addition of prochlorperazine as maintenance therapy. All of the cases were treated according to clinical guideline. Life expectancy of all patients was less than 3 months. Prochlorperazine was added with salvage therapy to prolong its effective duration from drug resistance. The aim of such maintenance therapy is to help control the disease without progression, allowing patients to live longer. The objective response rate (ORR), progression-free survival (PFS), and overall survival (OS) after prochlorperazine treatment showed that the use of maintenance therapy indeed effectively kept disease status stable and prolonged survivals. Median ORR was −6.2% (−79.8% ˜4.3%). Median PFS and OS after prochlorperazine treatment was 12.8 (7.0-20.1) months and 13.5 (7.4-21.4) months, respectively.

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

As used herein, the term “prochlorperazine” refers to a dopamine (D2) receptor antagonist that is used for the antiemetic treatment of nausea and vertigo. Prochlorperazine has the structure of

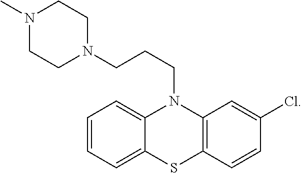 [SMILES: CN1CCN(CCCN2c3ccccc3Sc3ccc(Cl)cc32)CC1]

As used herein the term “pharmaceutically acceptable salt” refers to any pharmaceutically acceptable salt of prochlorperazine. The pharmaceutically acceptable salts include ammonium salts, alkali metal salts such as potassium and sodium (including mono, di-and tri-sodium) salts (which are preferred), alkaline earth metal salts such as calcium and magnesium salts, salts with organic bases such as dicyclohexylamine salts, N-methyl-D-glucamine, and salts with amino acids such as arginine, lysine, and so forth.

As used herein, the term “metabolite” refers to any intermediate and product of metabolism. Some examples of the metabolites of prochlorperazine include but are not limited to N-desmethyl prochlorperazine, prochlorperazine sulfoxide and prochlorperazine sulfoxide 4′-N-oxide. In one particular example, the metabolite is N-desmethyl prochlorperazine.

As used herein, the term “analog” refers to any compound with an altered chemical structure having the same function or activity. Some examples of the analog of prochlorperazine include but are not limited to the compounds having the following structures:

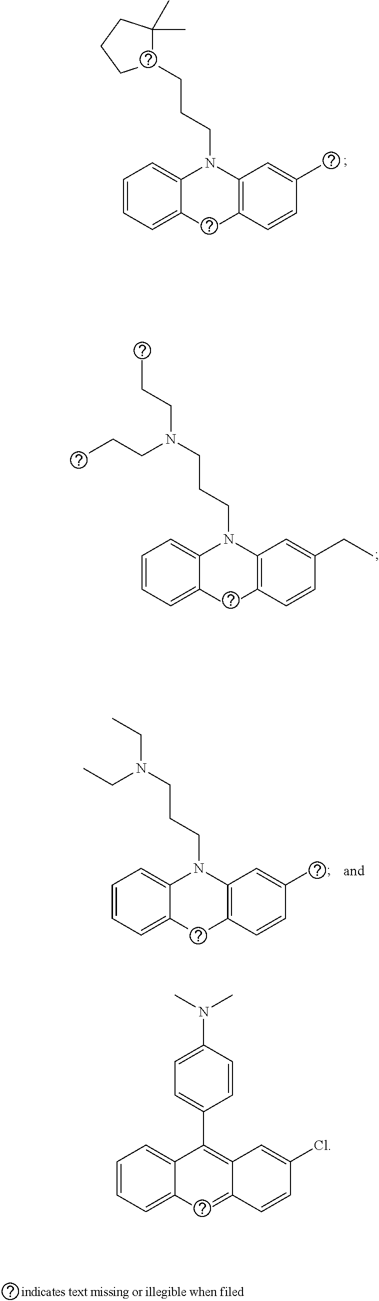

As used herein, the term “subject” refers to any warm-blooded species such as humans and animals. The subject, such as a human, to be treated according to the present invention may in fact be any subject of the human population, male or female, which may be divided into children, adults, or elderly. Any one of these patient groups relates to an embodiment of the invention.

The invention provides a method for treating a subject with a cancer resistant to a chemotherapeutic drug. The method comprises administering to said subject a therapeutically effective amount of prochlorperazine or its analog or metabolite, or a pharmaceutically acceptable salt thereof, in combination of the chemotherapeutic drug.

In some examples of the present invention, the combination of prochlorperazine and a chemotherapeutic drug exhibits a synergistic effect in reducing the size and number of the cancer cells. In other examples of the present invention, the combination of prochlorperazine and a chemotherapeutic drug exhibits a synergistic effect in inhibiting the growth of cancer cells.

As used herein, the term “chemotherapeutic drug” refers to any drug providing anti-cancer effect, including but not limited to gefitinib, erlotinib, afatinib, pemetrexed, cisplatin, paclitaxel, docetaxel, gemcitabine, navelbine, irinotecan, avastin, 5-fluorouracil, methotrexate, oxaliplatin, tegafur-gimeracil-oteracil potassium (TS-1), and epidermal growth factor receptor (EGFR)-tyrosine kinase inhibitors and combination thereof. Preferred examples include gefitinib, erlotinib, afatinib, pemetrexed, cisplatin, 5-fluorouracil, irinotecan, paclitaxel, gemcitabine, avastin, TS-1, oxaliplatin. In one particular example, the chemotherapeutic drug is gefitinib.

It was found in the examples of the invention that some metabolites have the same cytotoxic activities as prochlorperazine, as shown in Table 2.

It was also found in the examples of the invention that some analogs have the same clonogenic activities as prochlorperazine, as shown in Table 3.

As used herein, the term “therapeutically effective amount” refers to an amount sufficient for providing an effect in treatment for a cancer, which is depending on the mode of administration and the condition to be treated, including age, body weight, symptom, therapeutic effect, administration route and treatment time.

In the present invention, various cancers can be treated with the method according to the invention. In some embodiments, the cancer is a solid cancer such as a solid tumor. In other embodiments, the cancer is “liquid” cancer or a hematological cancer. The cancer is selected from the group consisting of lung cancer, liver cancer, colorectal cancer, brain cancer, breast cancer, pancreatic cancer, gastric cancer, adenocarcinoma, squamous cell carcinoma, and large cell carcinoma.

It is found in the example of the invention that prochlorperazine exhibited cytotoxicity in various types of cancers, including adenocarcinoma, squamous cell carcinoma, large cell carcinoma, liver cancer, colorectal adenocarcinoma, brain cancer, breast cancer, pancreatic cancer, and myeloma, see Table 1.

The present invention provides a method treating a subject with a cancer resistant to a chemotherapeutic drug. The method comprises administering to said subject a therapeutically effective amount of prochlorperazine or its analog or metabolite, or a pharmaceutically acceptable salt thereof, in combination of the chemotherapeutic drug. In one example of the invention, the caner is a lung cancer such as non-small cell lung carcinoma (NSCLC).

On the other hand, the present invention provides a method for preventing cancer metastasis. The method comprises administering to a subject in need thereof a therapeutically effective amount of prochlorperazine or its analog or metabolite, or a pharmaceutically acceptable salt thereof, in combination of a chemotherapeutic drug. In one example of the invention, the caner is a lung cancer such as lung squamous cell carcinoma. In another example of the invention, the cancer is gastric cancer, such as signet ring cell carcinoma.

In one example of the invention, prochlorperazine in combination of erlotinib provided an effect in prevention of metastasis of a lung cancer, such as lung squamous cell carcinoma. In another example of the invention, prochlorperazine in combination of a chemotherapeutic drug, which is selected from the group consisting of cisplatin, 5-fluorouracil, irinotecan, paclitaxel, gemcitabine, avastin, TS-1, oxaliplatin and combination thereof, provided an effect in prevention of metastasis of a gastric cancer, such as signet ring cell carcinoma.

In addition, the invention provides a use of prochlorperazine or its analog or metabolite, or a pharmaceutically acceptable salt thereof for manufacturing a medicament for treating a subject with a cancer resistant to a chemotherapeutic drug. The invention also provides a use of prochlorperazine or its analog or metabolite, or a pharmaceutically acceptable salt thereof for manufacturing a medicament for preventing cancer metastasis.

In the invention, prochlorperazine or its analog or metabolite, or a pharmaceutically acceptable salt thereof (“the active compound”) may be formulated in a pharmaceutical composition or formulation, which may be administered in any route that is appropriate, including but not limited to oral or parenteral administration. In one example of the invention, the composition or formulation comprising prochlorperazine or its analog or metabolite, or a pharmaceutically acceptable salt thereof is administered through oral route, which may he in a solid or liquid form. The solid compositions or formulations include tablets, pills, capsules, dispersible powders, granules, and the like. The oral compositions also include gargles which are to be stuck to oral cavity and sublingual tablets. The capsules include hard capsules and soft capsules. In such solid compositions or formulations for oral use, one or more of the active compound(s) may be admixed solely or with diluents, binders, disintegrators, lubricants, stabilizers, solubilizers, and then formulated into a preparation in a conventional manner. When necessary, such preparations may be coated with a coating agent, or they may be coated with two or more coating layers. On the other hand, the liquid compositions for oral administration include pharmaceutically acceptable aqueous solutions, suspensions, emulsions, syrups, elixirs, and the like. In such compositions, one or more of the active compound(s) may be dissolved, suspended or emulsified in a commonly used diluent (such as purified water, ethanol or a mixture thereof, etc.). Besides such diluents, said compositions may also contain wetting agents, suspending agents, emulsifiers, sweetening agents, flavoring agents, perfumes, preservatives and buffers and the like.

The pharmaceutical compositions for parenteral administration include solutions, suspensions, emulsions, and solid injectable compositions that are dissolved or suspended in a solvent immediately before use. The injections may be prepared by dissolving, suspending or emulsifying one or more of the active ingredients in a diluent. Examples of said diluents are distilled water for injection, physiological saline, vegetable oil, alcohol, and a combination thereof. Further, the injections may contain stabilizers, solubilizers, suspending agents, emulsifiers, soothing agents, buffers, preservatives, etc. The injections, are sterilized in the final formulation step or prepared by sterile procedure. The pharmaceutical composition of the invention may also he formulated into a sterile solid preparation, for example, by freeze-drying, and may be used after sterilized or dissolved in sterile injectable water or other sterile diluent(s) immediately before use.

The examples below are to be construed as merely illustrative, and not limitative of the remainder of the disclosure in any way whatsoever. Without further elaboration, it is believed that one skilled in the art can, based on the description herein, utilize the present invention to its fullest extent.

### Examples

I. Cell Culture and Chemicals

A549, CL141 and H441 are EGER-wild type adenocarcinoma cell lines, HCC827 has EGFR-exon 19 deletion, and CL97 is an EGFR T790M and G719A mutations cell line. CL152, H2170 and H226 are squamous cell carcinoma cell lines and H1299 is a non-small cell lung carcinoma cell line. A549-ON cell line is A549 cell overexpression Oct4 and Nanog which we regarded it as cancer stem cell-like cell line (22). All cell lines were maintained in RPMI medium and supplemented with 10% fetal bovine serum (FBS, Invitrogen), 2 mM L-glutamine, 100 U/mL penicillin, and 100 μg/mL streptomycin. For cell culture experiment, 10 mM prochlorperazine stock solution, was dissolved in dimethyl sulfoxide (DMSO; Sigma).Prochlorperazine, Cisplatin, Gemcitabine were purchased from Sigma.

II. Cytotoxicity and Sulforhodamine B Assay

Cells were plated in 96-well plates at a density of 2000 cells per well in triplicate. The cells were treated on the third day (to ensure proper plating efficiency and vitality) to indicated agents for 48 hrs. Cells were treated with different concentrations of prochlorperazine, cisplatin, gemcitabine, or a combination of, for example, prochlorperazine and gemcitabine. Cytotoxicity was assessed using the sulforhodamine B (SRB) assay (23). Briefly, the medium was discarded, and the adherent cells were fixed by 100 μl of cold 10% trichioroacetic acid (w/v) in each well for 1 h at 4° C. After fixation, cells were stained with 100 μl/well of 0.4% (w/v, in 1% acetic acid) SRB solution for 30 min at room temperature, and then washed 5 times with 1% acetic acid. After air-drying, 100 μl of 10 mM Tris base was added to each well and the absorbance was read at 530 nm. Cytotoxicity is expressed as the percent of cells in drug treated wells relative to number of cells in the solvent only control (set to 100%). Each experiment was performed independently at least 2 times in triplicate and cytotoxicity is given as means ±SD.

III. Clonogenic Assay

Tested cells were seeded respectively in 6 well plates with 10⁴ cells per well for 14 days. Prochlorperazine and other tested drugs were added 24 hours after seeding of the cells. The medium and prochlorperazine were changed every 4 days. After the treatments, cells were washed with PBS, and the colonies were fixed with fix solution (methanol: acetic acid=3:1) and stained with 0.5% crystal violet in methanol. After removing the crystal violet carefully and rinse with tap water, the colonies were counted manually. Each experiment was performed independently at least 2 times in triplicate and cytotoxicities are given as means ±SD.

IV. Tumor Spheroid Assay

In brief, single cells were plated in 6-well ultralow attachment plates (Coming Inc.) at a density of 2,000 cells/mL in tumor spheroid culture medium, DMEM/F12 supplemented with 1% N2 Supplement (Invitrogen), 10 ng/mL basic fibroblast growth factor (Sigma-Aldrich), 10 ng/mL epidermal growth factor (Invitrogen) with 1% penicillin/streptomycin (Invitrogen) at 37° C. in a humidified atmosphere of 95% air and 5% CO2. Cells were cultured twice per week. When passaged, tumor spheres were harvested. Spheroids were dissociated with TrypLE™ (Invitrogen). Spheroids cell counting using the Trypan Blue Exclusion method.

V. Side Population Analysis and Purification Using Flow Cytometry

Single-cell suspensions of cells were detached from dishes with Trypsin-EDTA (Invitrogen) and suspended at 1×10⁶ cells/mL in Hank's balanced salt solution (HIM) supplemented with 3% fetal calf serum and 10 mM Hepes. These cells were then incubated at 37° C. for 90 minutes with 20 μg/mL Hoechst 33342 (Sigma Chemical, St. Louis, Mo.). The ABC transporter inhibitor verapamil (Sigma) was added at a final concentration of 50 μM to confirm the gating area on flow cytometry. After 90 minutes incubation with indicated drugs, the cells were centrifuged immediately for 5 minutes at 300 g and 4° C. and resuspended in ice-cold HBSS, The cells were kept on ice to inhibit efflux of the Hoechst dye, and 1 μg/mL propidium iodide (PI, BD) were added to discriminate dead cells. Finally, these cells were filtered through a 40 μm cell strainer (BD) to obtain single-suspension cells. Cell dual-wavelength analysis and purification were performed on a dual-laser FACS Vantage SE (BD). Hoechst 33342 can be excited at 355 nm UV light and emitted blue fluorescence with a 450/20 band-pass (BP) filter and red fluorescence with a 675 nm edge filter long-pass (EFLP). A 610 nm dichroic mirror short-pass (DMSP) was used to separate the emission wavelengths. PI-positive (dead) cells were excluded from the analysis.

VI. Aldefluor Assay

High aldehyde dehydrogenase (ALDH) enzyme activity was used to detect ung cancer stem cell populations. The Aldefluor assay was performed according to the manufacturer's guidelines (StemCell Technologies). Briefly, single cells obtained from cell cultures were incubated in an Aldefluor assay buffer containing an ALDH substrate (bodipy-aminoacetaldehyde, BAAA) for 50 minutes at 37° C. As a negative control, a fraction of cells from each sample was incubated under identical conditions in the presence of an ALDH inhibitor (diethylaminobenzaldehyde, DEAB). Flow cytometry was used to measure the ALDH-positive cell population.

VII. In Vivo Examination of Anti-Lung Cancer Effects Mediated by Prochlorperazine.

In the first trial, human lung cancer cell line NCI-H441 (purchased from ATCC, 1 million cells/injection) cells were subcutaneously injected into the right flank of NOD/SCID mice (female, 4-6 weeks old). When tumors became palpable, their sizes were recorded using caliper and mice were randomly divided into control group (DMSO vehicle) and prochlorperazine treatment group (5 mg/kg, 5 days/week, i.p injection). Over the period of 4 weeks, tumorigenesis in both groups was measured using a caliper on a weekly basis. The change in tumor size was expressed as in fold change and plotted over time. Prochlorperazine treatment appeared to suppress and/or delay the growth of tumor as compared to the vehicle control (**p<0.01).

In the first drug combination test, NCI-H441 cells expressing firefly luciferase (6×10⁵ cells/injection) were injected into NOD/SCID mice (4-6 week of age) via the lateral tail vein for tumor establishment. One week post tumor injection, mice were randomly divided into different groups: vehicle, prochlorperazine (1 mg/kg) in combination with pemetrexed (1 mg/kg) (a standard chemotherapeutic agent for NSCLC), pemetrexed (1 mg/kg)+cisplatin (1 mg/kg) and pemetrexed (1 mg/kg)+prochlorperazine (1 mg/kg) treatment groups. Tumor burden from different groups were recorded by caliper. The change in tumor size was expressed as in fold change and plotted over time. Subsequently, we performed another set of drug combination test. We used two major standard chemotherapeutic regimens in combination with prochlorperazine. For lung adenocarcinoma, the standard drug combination is pemetrexed and cisplatin whereas are used gemcitabine and cisplatin for lung squamous carcinoma. In CL97 (adenocarcinoma) tumor-bearing mice, the combination of pemetrexed+cisplatin+prochlorperazine provided the most significant tumor suppressive effect than pemetrexed+cisplatin and prochlorperazine alone groups. In CL152 (squamous) tumor-bearing mice, again standard regimen (gemcitabine+cisplatin) added with prochlorperazine appeared to suppress tumorigenesis better than prochlorperazine alone and gemcitabine+cisplatin groups. Next, we demonstrated that prochlorperazine sensitized gefitinib-resistant CL97 lung cancer cells in vivo. CL97 cancer cells (1.5×10⁶ cells per injection) were injected subcutaneously into NOD/SCID mice to establish in viva tumor model. Mice were randomly divided into 4 groups: Control, gefitinib alone (100 mg/kg, PO, 5 times/week), prochlorperazine alone (5 mg/kg, IP, 5 times/week) and gefitinib+prochlorperazine groups. Significant tumor suppressive effect exerted by prochlorperazine alone and gefitinib +prochlorperazine groups were observed 5-week post tumor injection. By week 6 post tumor injection, gefitinib+prochlorperazine group showed the most significant tumor suppressive effect followed by prochlorperazine alone group while both control and gefitinib alone groups demonstrated a similar tumor burden.

Results

Cytotoxicity of Prochlorperazine in NSCLC Cell Lines

We first investigated the cytotoxic effect of prochlorperazine in various NSCLC cell lines. A549, A549-ON, CL97, CL141, HCC827, and H441 were lung adenocarcinoma cell lines. CL152, H2170, and H226 were squamous cell carcinoma cell lines. Cell viability was determined by SRB assay. After treatment for 48 hours with prochlorperazine with various concentrations, the IC50 values in all cancer cell lines were around 20 μM (see Table 1). The experiments were perfoinied to determine the anti-tumor activities of prochlorperazine using clonogenic assay. Clonogenicity of NSCLC cell lines was reduced in a concentration-dependent manner after exposure to different concentration of prochlorperazine Cancer stem cell (CSC) postulates the existence of a tumor cell population uniquely endowed with self-renewal capacity and therapy resistance. Since prochlorperazine was predicted to preferentially inhibit the lung CSC, we next examined whether the proportion of NSCLC (a lung squamous cell carcinoma cell with wild-type EGFR) spheres could be diminished by prochlorperazine treatment. To determine whether side population (SP) cells, which represent a cell population with CSC characteristics, exist in NSCLC cell lines, we stained the cells with the fluorescent dye, Hoechst 33342, and analyzed the cells with flow cytometry. After the exclusion of dead cells and cellular debris based on the scatter signals, the NSCLC cells contained a small population of cells with SP cell characteristics. After 48 hours of incubation with prochlorperazine at 2.5, 5 and 10 μM, the proportion of SP cells were dose-dependently decease (see Table 1).

Prochlorperazine reduces the proportion of side population cells and ALDH⁺ cells

It was also investigated whether prochlorperazine treatment could deplete the percentage of the cells with ALDH expression (ALDH is an established marker for both hematopoietic and NSCLC CSCs). As shown in Table 1, prochlorperazine treatment also decreased the ALDH⁺ CL152 population in a dose-dependent manner. In conclusion, prochlorperazine showed low or minimal cytotoxic effects in NSCLC cells.

TABLE 1
Cytotoxcities of Prochlorperazine to Various Types of Cancers

| | | | IC50 for | Clonogenic | Sphere | Side | ALDH⁺ |
|---|---|---|---|---|---|---|---|
| | | EGFR mutation | prochlorperazine | assay | formation | population | assay |
| Cancer type | Cell line | status | (μM) | (μM) | (μM) | (μM) | (μM) |
| Adenocarcinoma | A549 | WT | 21.5 ± 1.1 | 5-10 | | | <5 |
| | A549-ON | WT | 21 ± 1.4 | | | | |
| | (overexpression | | | | | | |
| | Oct4 and Nanog) | | | | | | |
| | CL97 | G719A/T790M | 13 ± 1.04 | 5-10 | 0-2.5 | <5 | <5 |
| | CL141 | WT | 12.7 ± 1 | 5-10 | 0-2.5 | >10 | <5 |
| | HCC827 | Exon 19 deletion | >20 | | 5-10 | | |
| | H441 | WT | 17.7 ± 1.3 | | 0-2.5 | <5 | <10 |
| Squamous cell | CL152 | WT | 16.6 ± 2.4 | 5-10 | 0-5 | <5 | <5 |
| carcinoma | H2170 | WT | 24.7 ± 0.4 | | | | |
| | H226 | WT | 21.2 ± 1.l | | 0-5 | | |
| Large cell carcinoma | H1299 | WT | 27.6 ± 8.8 | 5-10 | 0-5 | | |
| Liver cancer | Mahlavu | | >10 | | >5 | | |
| | SK-Hep 1 | | >20 | | >5 | | |
| | HepG2 | | >10 | | | | |
| Colorectal | HT29 | | 13.5 ± 0.05 | | 0-2.5 | | |
| adenocarcinoma | | | | | | | |
| Brain cancer | GBM8401 | | ~10 | 3.33-10 | | >10 | |
| | U87MG | | | | 10-15 | >10 | |
| Breast cancer | MDA-MB-231 | | | | 5-10 | | |
| Pancreatic cancer | PANC-1 | | | | 10-20 | | |
| Myeloma | J5 | | | | >5 | | |

In addition, the cytotoxicities of prochlorperazine metabolites at NSCLC parental and sphere cells were examined by SRB assay and Trypan Blue Exclusion method. It was found that N-desmethyl prochlorperazine showed better cytotoxic effect than other tested metabolites in NSCLC cell lines (see Table 2).

TABLE 2
IC₅₀ for the Metabolites of Prochlorperazine
IC₅₀ for prochlorperazine metabolites (μM)

| | | | | |
|---|---|---|---|---|
| | | N-desmethyl | Prochlorperazine | Prochlorperazine |
| Compound | Prochlorperazine | prochlorperazine | sulfoxide | sulfoxide 4′-N-oxide |
| Structure | 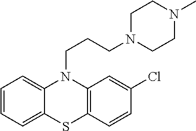 [SMILES: CN1CCN(CCCN2c3ccccc3Sc3ccc(Cl)cc32)CC1] | 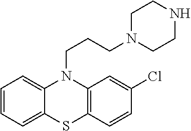 [SMILES: Clc1ccc2c(c1)N(CCCN1CCNCC1)c1ccccc1S2] | 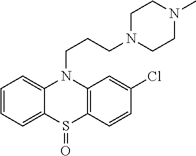 [SMILES: CN1CCN(CCCN2c3ccccc3S(=O)c3ccc(Cl)cc32)CC1] | 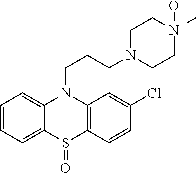 [SMILES: C[N+]1([O-])CCN(CCCN2c3ccccc3S(=O)c3ccc(Cl)cc32)CC1] |
| CL141 | 10-20 | 5-10 | >20 | >20 |
| CL152 | 10-20 | ~10 | >20 | >20 |
| CL141 | <5 | <5 | 5-10 | >10 |
| spheres | | | | |
| CL152 | <5 | <5 | >10 | >10 |
| spheres | | | | |

Also, the effect of the analogs of prochlorperazine on the clonogenicity in CL141 were examined, wherein approximately 1,000 cells were seeded in 6-well plate and treated with different analogs; after 14 days, the colonies were stained with crystal violet, photographed and counted. The results are shown in Table 3, showing that four analogs of prochlorperazine reduced clonogenicity in NSCLC cells.

TABLE 3
Clonogenic assay for the Analogs of Prochlorperazine
Clonogenic assay for prochlorperazine analogs (μM)

| Compound | NSC 14948 | NSC 166184 | NSC 169470 | NSC 406619 |
|---|---|---|---|---|
| Structure | 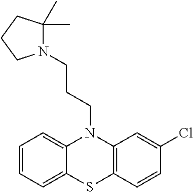 [SMILES: CC1(C)CCCN1CCCN1c2ccccc2Sc2ccc(Cl)cc21] | 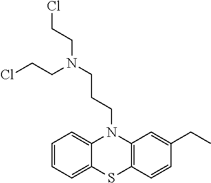 [SMILES: CCc1ccc2c(c1)N(CCCN(CCCl)CCCl)c1ccccc1S2] | 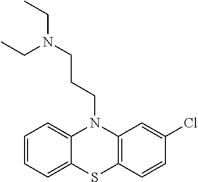 [SMILES: CCN(CC)CCCN1c2ccccc2Sc2ccc(Cl)cc21] | 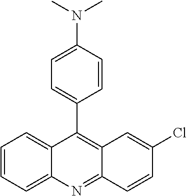 [SMILES: CN(C)c1ccc(-c2c3ccccc3nc3ccc(Cl)cc23)cc1] |
| CL141 | 5-10 | 1-2.5 | 5-10 | 2.5-5 |

Prochlorperazine induces apoptosis in CL152 spheres and synergistically enhances cytotoxicity in combine with gemcitabine.

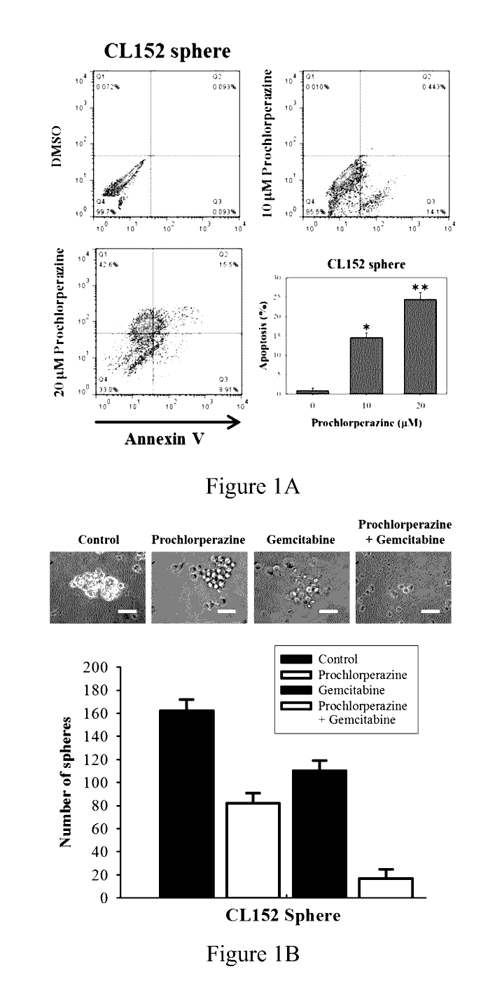

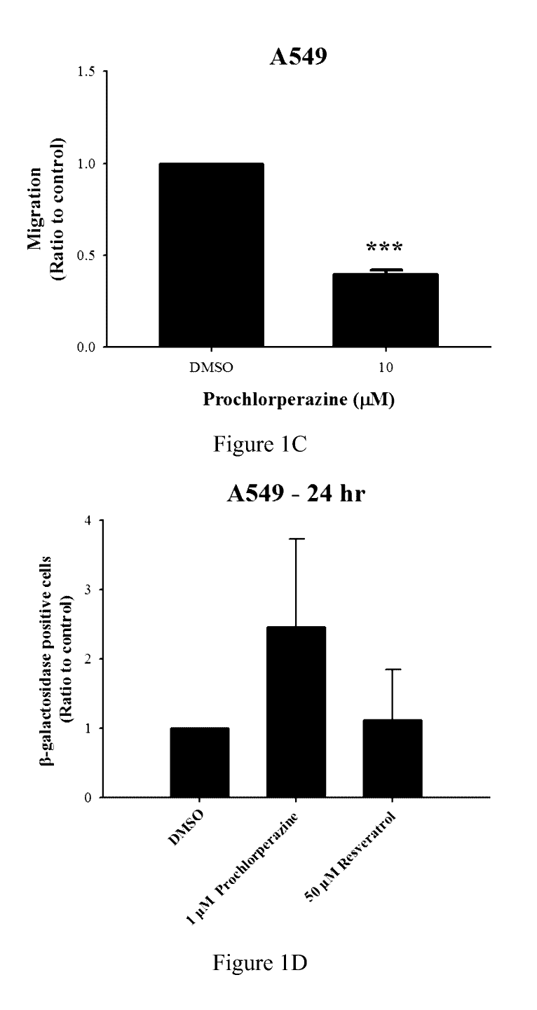

After treatment with prochlorperazine for 48 hours, CL152 spheres were collected and analyzed by flow cytometry. Prochlorperazine led to a dose-dependent increase in apoptosis (FIG. 1A). Gemcitabine is a chemotherapeutic agent frequently used in NSCLC treatment. Next, the combination effect of prochlorperazine and gemcitabine was investigated by sphere-formation assay. The result demonstrated that prochlorperazine showed synergistic effect with genicitabine in CL1.52 spheres (FIG. 1B).

Prochlorperazine inhibits lung cancer cell migration and induces senescence

Cancer cells undergoing epithelial-mesenchymal transition (EMT) have been found to show increased resistance to apoptosis and certain chemotherapeutic drugs. EMT also plays a critical role in the regulation of cancer sternness. EMT is characterized by enhanced cell migration and invasion. Transwell migration assay was used to determine whether sub-cytotoxic concentration of prochlorperazine could inhibit cell motility of A549 cells in vitro. After 24 hours, treatment with 10 μM prochlorperazine significantly inhibited cell migration of A549 cells as compared with the DMSO controls (FIG. 1C). Furthermore, we examined the effect of prochlorperazine on the senescence at A549 cells. β-galactosidase (SA-β-Gal) activity was detected by senescence detection kit (BioVision Inc.). After treatment with 1 μM prochlorperazine and 50 μM resveratrol as positive control for 24 hours, A549 cells increased β-galactosidase activity and induced β-galactosidase activity positive cells were counted by microscope at 200x field (FIG. 1D). These data showed that prochlorperazine could induce senescence at NSCLC cells at a low concentration.

Prochlorperazine significantly inhibits the self-renewal of NSCLC cancer spheres.

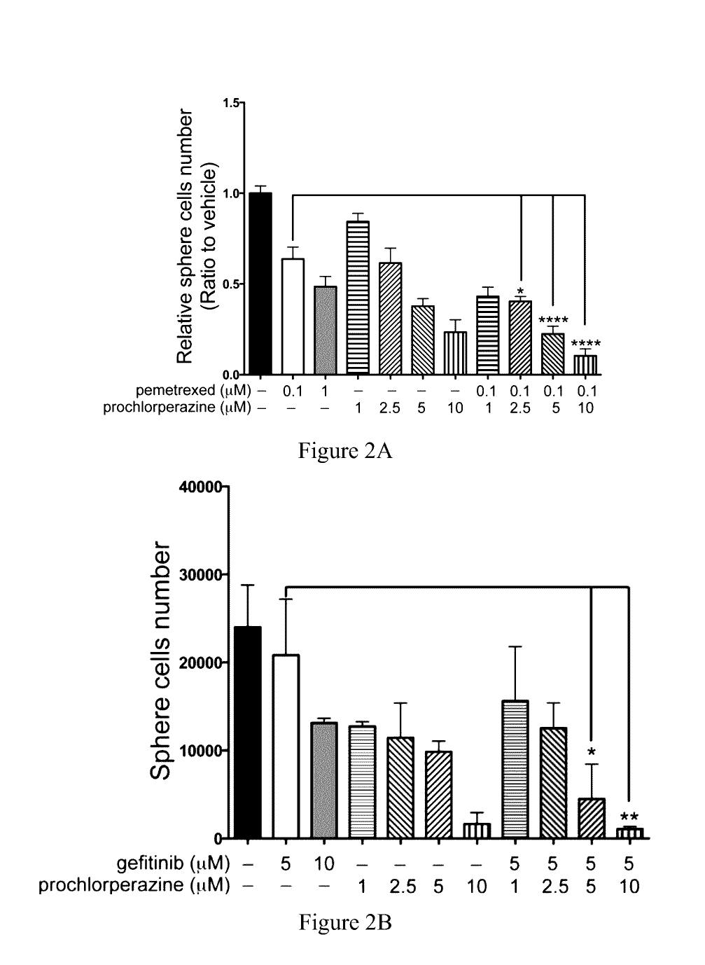

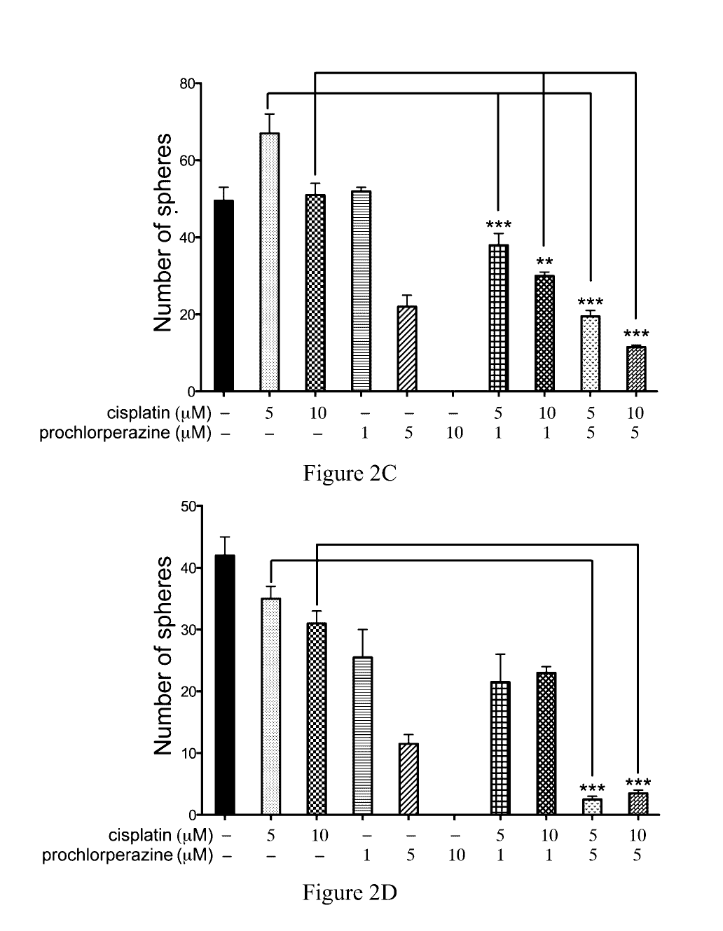

To evaluate whether the prochlorperazine alone or combined with clinical drugs, chemotherapeutic agents and EGFR-tyrosine kinase inhibitors gefitinib, treatment could suppress NSCLC cancer stem-like sphere cells in vitro. CL141 sphere cells (FIG. 2A) and CL97 sphere cells (FIG. 2B) were treated with prochlorperazine alone, pemetrexed (CL141 spheres) or gefitinib (CL97 spheres) alone, or a combined treatment for 48 hours. Prochlorperazine (1, 2.5, 5 and 10 μM) reduced the number of spheres from the CL141 and CL97 cells in a concentration-dependent manner (FIG. 2A and 2B). The combination, co-treatment with prochlorperazine and clinical drugs, also significantly decreased the number of sphere cells compared to pemetrexed or gefitinib alone (FIG. 2A and 2B). To further explore the effect of prochlorperazine combine with cisplatin in CSC, we established the sphere-formation assay of two NSCLC cell lines, including HCC827 and H1299. Prochlorperazine suppressed the sphere-forming ability (FIG. 2C and 2D). Interestingly, the combination with cisplatin seemed to have lower spheres number formed by these NSCLC cells than that of cells treated with prochlorperazine or cisplatin alone. Taken together, these data suggest that prochlorperazine has anti-CSC ability on these tested spheres and combination of prochlorperazine with chemotherapeutic agents or EGFR-tyrosine kinase inhibitors may have benefited to cancer therapy.

In vivo examination of tumor inhibitory effects of prochlorperazine.

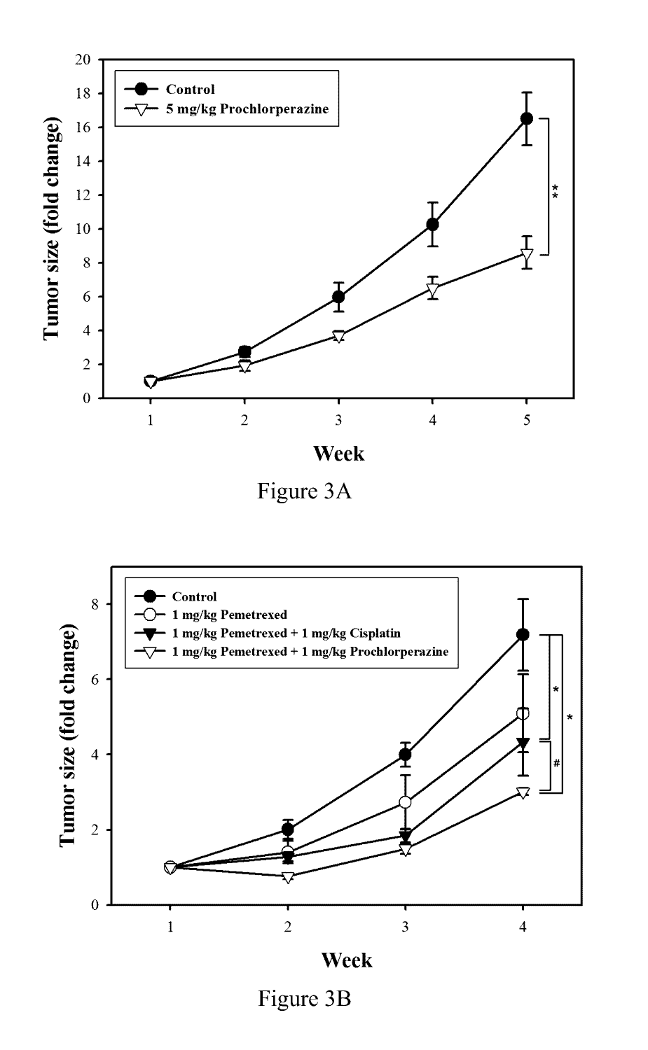

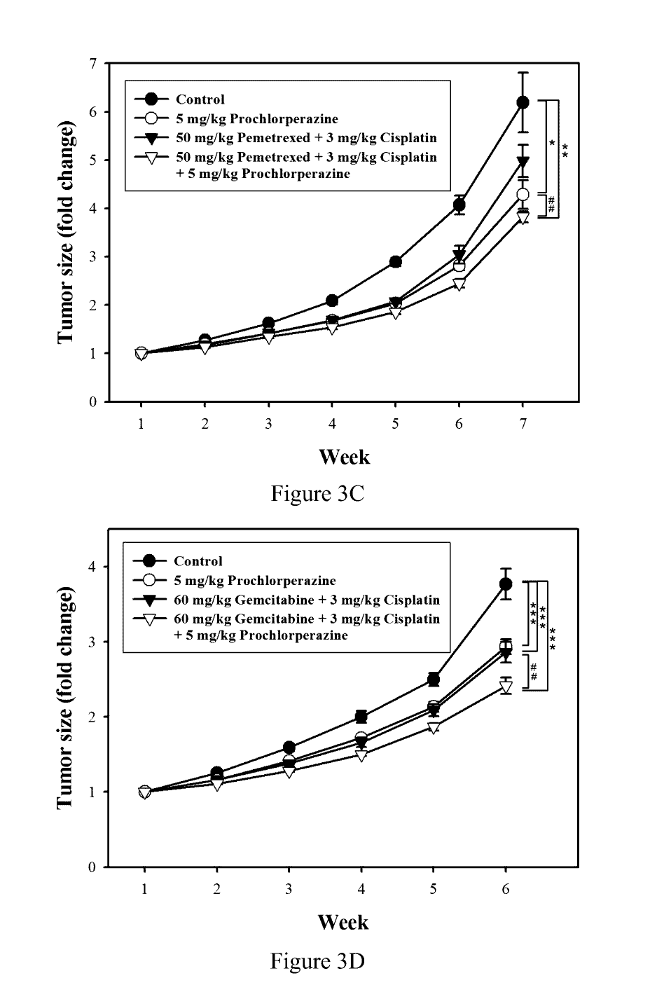

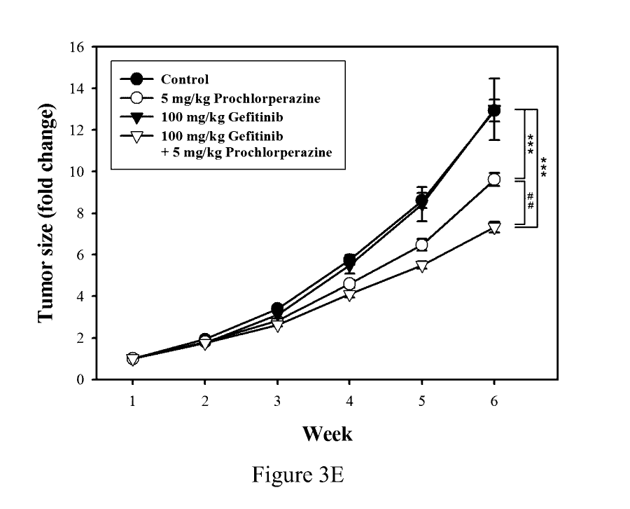

In the first trial (FIG. 3A), NCI-H441 (1×10⁶ cells/injection) cells were subcutaneously injected into the right flank of NOD/SCID mice (female, 4-6 weeks old). When tumors became palpable, their sizes were recorded using caliper and mice were randomly divided into control group (DMSO vehicle) and prochlorperazine treatment group (5 mg/kg, 5 days/week, i.p. injection). Four weeks post treatment, it was clear that prochlorperazine, at 5 mg/kg, was effective in suppressing tumor growth as compared to the vehicle controls (**p<0.01).

In the drug combination test (FIG. 313), NCI-H441 cells expressing firefly luciferase (6×10³ cells/injection) were injected into NOD/SCID mice (4-6 week of age) via the lateral tail vein for tumor establishment. One week post tumor injection, mice were randomly divided into different groups: control, prochlorperazine (1 mg/kg) in combination with pemetrexed (1 mg/kg) (a standard chemotherapeutic agent for NSCLC), the combination of pemetrexed (1 mg/kg) and cisplatin (1 mg/kg), and the combination of pemetrexed (1 mg/kg) and prochlorperazine (1 mg/kg) groups. Interestingly, we found that using our dosing regimen, the inhibitory effect on tumor growth by cisplatin (at 1 mg/kg) in combination with pemetrexed was not significantly different from that in the pemetrexed alone (1 mg/kg) group (see FIG. 3B). However, the combination of prochlorperazine and pemetrexed provided a significantly better tumor inhibitory effect than any other groups (*p<0.05).

In other drug combination tests, CL97 (FIG. 3C) and CL152 (FIG. 3D) cells were subcutaneously injected into the right flank of NOD/SCID mice (female, 4-6 weeks old). One week post tumor injection, mice were randomly divided into different groups: control group (DMSO vehicle) and prochlorperazine treatment group (5 mg/kg), standard treatment group (50 mg/kg pemetrexed or 60 mg/kg gemcitabine combined with 3 mg/kg cisplatin), and the combination group (standard treatment combined with 5 mg/kg prochlorperazine). From the animal data, prochlorperazine alone could inhibit tumor growth (*p<0.05, ***p<0.001); standard treatment combined with prochlorperazine showed more significantly inhibitory effect of tumor growth than other treatments (**p<0.01, ***p<0.001).

To examine whether prochlorperazine could overcome the drug resistance, CL97 (EGFR T790M and G719A mutations) cells were subcutaneously injected into the right flank of NOD/SCID mice (female, 4-6 weeks old). One week post tumor injection, mice were randomly divided into different groups: control group (DMSO) vehicle) and prochlorperazine treatment group (5 mg/kg), target-therapy group (100 mg/kg gefitinib), and combination group (100 mg/kg gefitinib combined with 5 mg/kg prochlorperazine). In FIG. 3E, gefitinib alone was ineffective to inhibit tumor growth. However, prochlorperazine alone or combined with gefitinib showed significantly inhibitory effects of tumor growth (***p<0.001). From the evidence, prochlorperazine could overcome the drug resistance through sensitizing chemotherapy or target therapy. This finding suggests that prochlorperazine could be considered as a clinical adjuvant therapeutic agent with chemotherapy or target therapy in the future.

Clinical Cases After Prochlorperazine Treatment

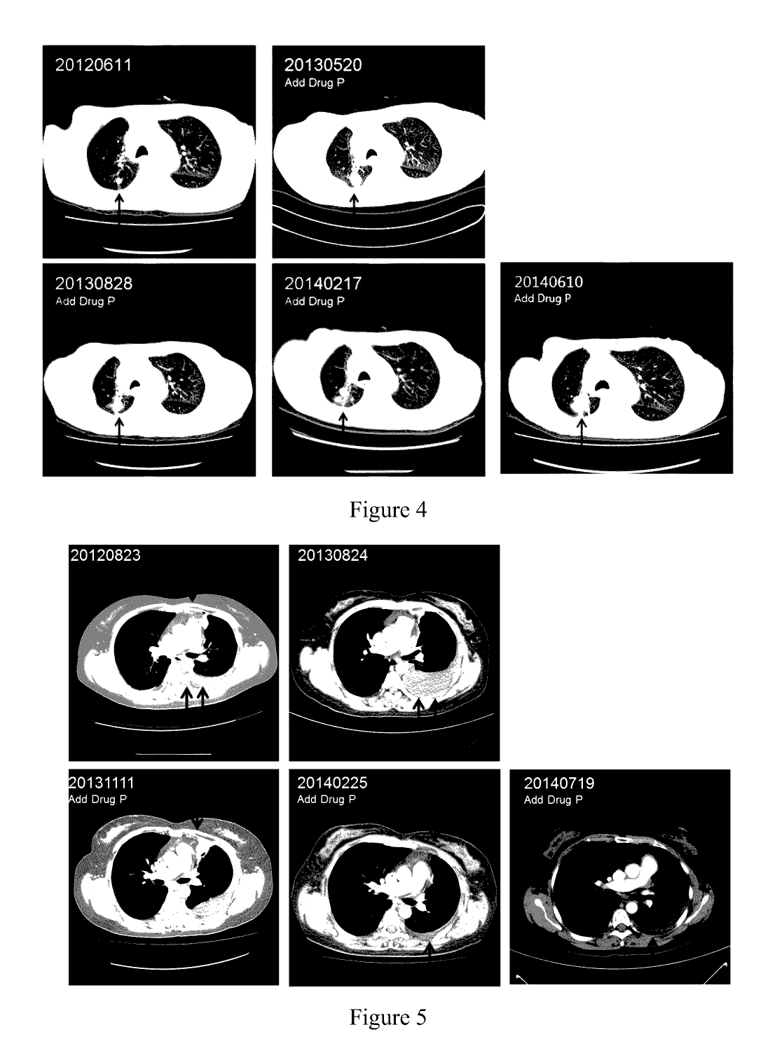

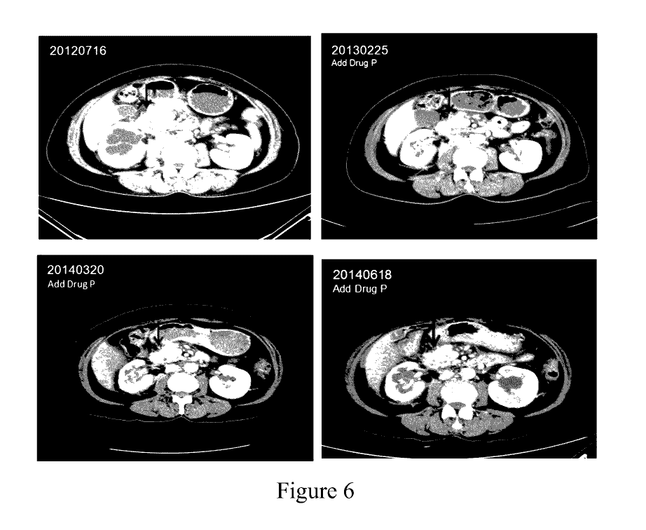

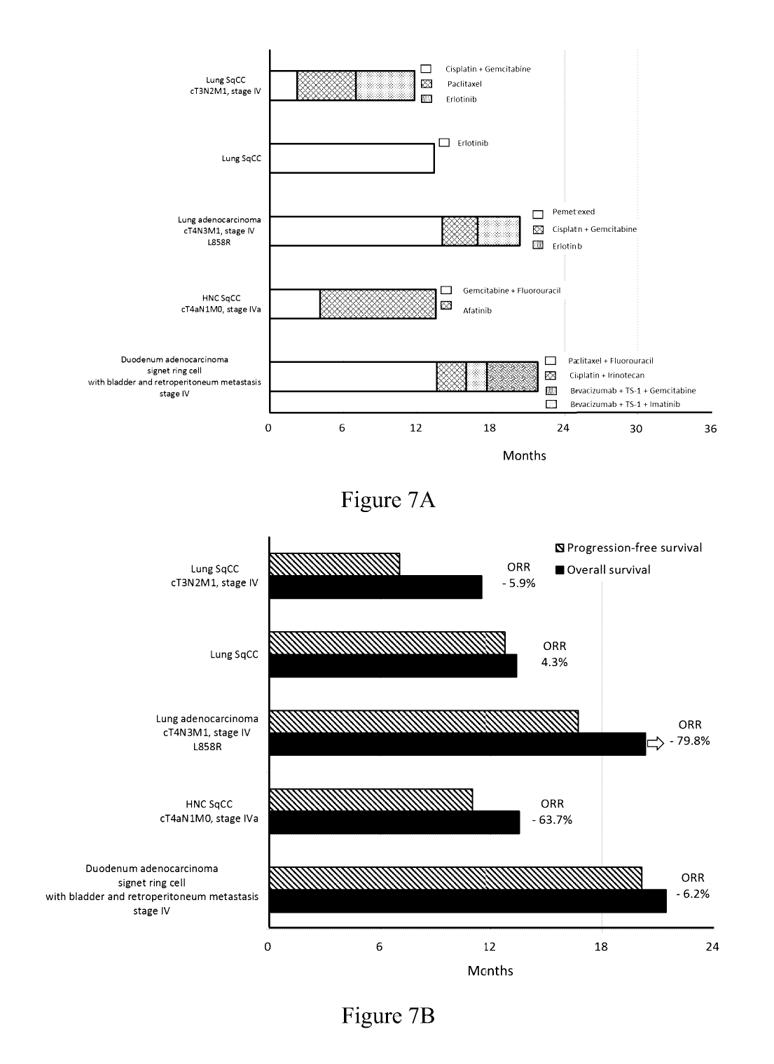

To prove the concept of the invention, clinical observation study of prochlorperazine (label as drug P in the figures) was performed (see FIGS. 4-6). The patients include lung squamous cell carcinoma patient treated with Tarceva™ (containing erlotinib as active ingredient) (as shown in FIG. 4), lung adenocarcinoma patient treated with pemetrexed (as shown in FIG. 5), and signet ring cell carcinoma patient treated with a combination of several kinds of chemotherapy, including cisplatin, 5-fluorouracil, irinotecan, paclitaxel, gemcitabine, avastin, TS-1, oxaliplatin, paclitaxel/5-fluorouracil and avastin/gemcitabine/TS-1 (as shown in FIG. 6), who also took prochlorperazine. It was observed that the patiens were in a stable or tumor reduction condition. The observations suggested that the addition of prochlorperazine in the current treatment has the benefits to the patients. In other cases, several patients took prochlorperazine for more than 1 year, suggesting the current dose could be tolerated with minimal side effect. Moreover, the addition of prochlorperazine could be considered as a maintenance therapy that helps the patients to control the disease without cancer progression, allowing the patients to live longer. In view of the data including median Objective response rate (ORR), progression-free survival (PFS), and overall survival (OS) after prochlorperazine treatment, the disease status of the patients was effectively kept stable and the prolonged survivals of the patients were observed (FIGS. 7A and 7B).

It can be concluded that prochlorperazine in combination with a chemotherapy might provide treatment benefits to patients with various cancers, particularly to overcome the drug resistant properties and to prevent cancer metastasis.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

I/We claim:

1. A use of prochlorperazine or its analog or metabolite, or a pharmaceutically acceptable salt thereof, for manufacturing a medicament for treating a subject with a cancer resistant to a chemotherapeutic drug, in combination of the chemotherapeutic drug.

2. The use of claim 1, wherein the chemotherapeutic drug is selected from the group consisting of gefitinib, erlotinib, afatinib, pemetrexed, cisplatin, paclitaxel, docetaxel, gemcitabine, navelbine, irinotecan, a:vastin, 5-fluorouracil, methotrexate, oxaliplatin, tegafur-gimeracil-oteracil potassium (TS-1), and epidermal growth factor receptor (EGFR)-tyrosine kinase inhibitors and combination thereof.

3. The use of claim 2, wherein the chemotherapeutic drug is selected from the group consisting of gefitinib, erlotinib, afatinib, pemetrexed, cisplatin, 5-fluorouracil, irinotecan, paclitaxel, gemcitabine, avastin, TS-1, oxaliplatin. and combination thereof.

4. The use of claim 2, wherein the chemotherapeutic drug is gefitinib

5. The use of claim 1, wherein the cancer is selected from the group consisting of lung cancer, liver cancer, colorectal cancer, brain cancer, breast cancer, pancreatic cancer, gastric cancer, adenocarcinoma, squamous cell carcinoma, and large cell carcinoma.

6. The use of claim 1, wherein the cancer is lung cancer.

7. The use of claim 6, wherein the lung cancer is non-small cell lung carcinoma (NSCLC).

8. The use of claim 1, wherein the metabolite of prochlorperazine is selected from the group consisting of N-desmethyl prochlorperazine, prochlorperazine sulfoxide and prochlorperazine sulfoxide 4′-N-oxide.

9. The use of claim 1, wherein the metabolite of prochlorperazine is N-desmethyl prochlorperazine.

10. The use of claim 1, wherein the analog of prochlorperazine is selected from the group consisting of the compounds of the following structures:

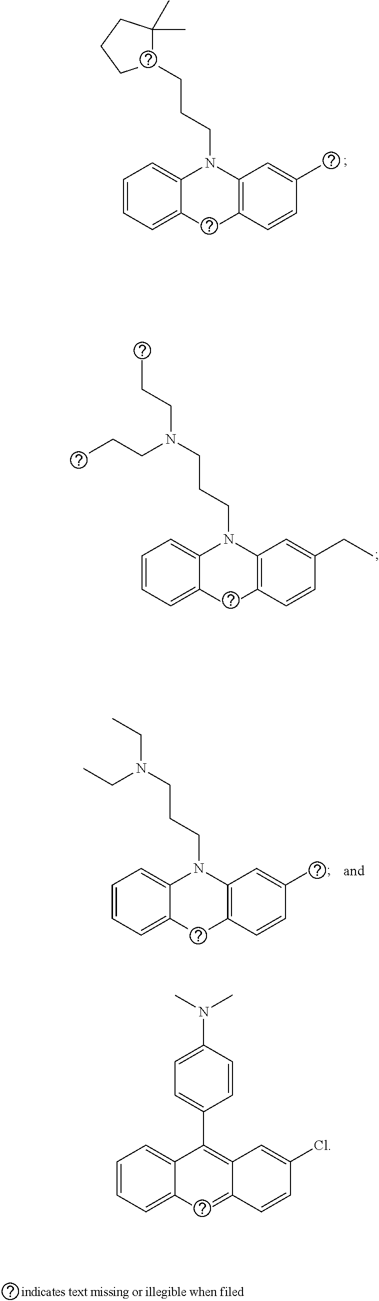

11. The use of claim 1, wherein the combination of prochlorperazine or its analog or metabolite, or a pharmaceutically acceptable salt thereof and the chemotherapeutic drug exhibits a synergistic effect in reducing the size and number of the cancer cells.

12. The use of claim 1, wherein the combination of prochlorperazine or its analog or metabolite, or a pharmaceutically acceptable salt thereof and the chemotherapeutic drug exhibits a synergistic effect in inhibiting the growth of cancer cells.

13. A use of prochlorperazine or its analog or metabolite, or a pharmaceutically acceptable salt thereof, for manufacturing a medicament for preventing cancer metastasis, in combination of a chemotherapeutic drug.

14. The use of claim 13, wherein the metabolite of prochlorperazine is selected from the group consisting of N-desmethyl prochlorperazine, prochlorperazine sulfoxide and prochlorperazine sulfoxide 4′-N-oxide.

15. The use of claim 14, wherein the metabolite of prochlorperazine is N-desmethyl prochlorperazine.

16. The use of claim 13, wherein the analog of prochlorperazine is selected from the group consisting of the compounds of the following structures:

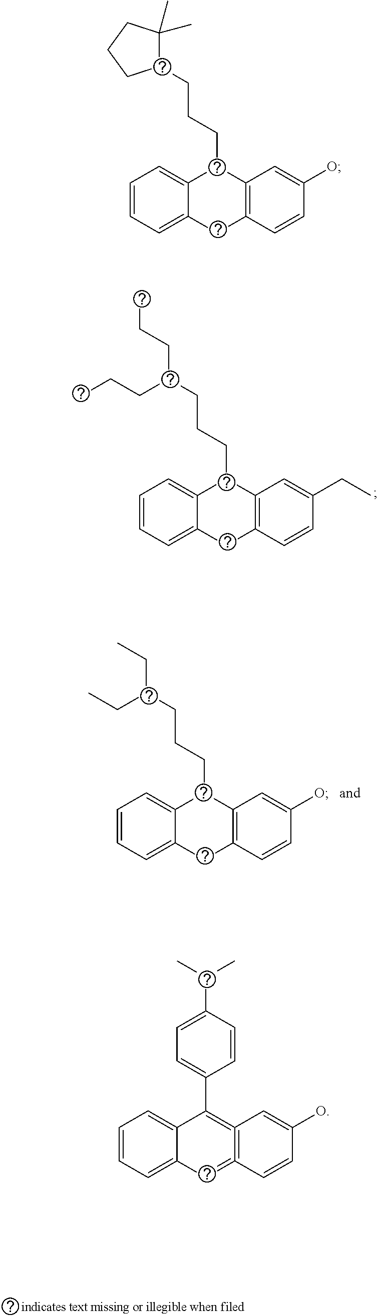

17. The use of claim 13, wherein the chemotherapeutic drug is selected from the group consisting of gefitinib, erlotinib, afatinib, pemetrexed, cisplatin, paclitaxel, docetaxel, gemcitabine, navelbine, irinotecan, avastin, 5-fluorouracil, methotrexate, oxaliplatin, TS-1, EGFR-tyrosine kinase inhibitors, and combination thereof.

18. The use of claim 13, wherein the cancer is selected from the group consisting of lung cancer, liver cancer, colorectal cancer, brain cancer, breast cancer, pancreatic cancer, gastric cancer, adenocarcinoma, squamous cell carcinoma, and large cell carcinoma.

19. The use of claim 13, wherein the cancer is lung cancer.

20. The use of claim 19, wherein the lung cancer is lung squamous cell carcinoma.

21. The use of claim 20, wherein the chemotherapeutic drug is erlotinib.

22. The use of claim 13, wherein the cancer is gastric cancer.

23. The use of claim 22, wherein the gastric cancer is signet ring cell carcinoma.

24. The use of claim 22, wherein the chemotherapeutic drug is selected from the group consisting of cisplatin, 5-fluorouracil, irinotecan, paclitaxel, gemcitabine, avastin, TS-1, oxaliplatin and combination thereof.
